(19) United States
(12) Patent Application Publication
(10) Pub. No.: US 2024/0382819 A1
(43) Pub. Date: Nov. 21, 2024

(54) GOLF LESSON SYSTEM USING TACTILE STIMULATION AND GOLF LESSON METHOD USING THE SAME

(71) Applicant: RESEARCH & BUSINESS FOUNDATION SUNGKYUNKWAN UNIVERSITY, Suwon-si (KR)

(72) Inventors: Hangue PARK, Suwon-si (KR); Jinhyoung PARK, Suwon-si (KR)

(73) Assignee: RESEARCH & BUSINESS FOUNDATION SUNGKYUNKWAN UNIVERSITY, Suwon-si (KR)

(21) Appl. No.: 18/667,345

(22) Filed: May 17, 2024

(30) Foreign Application Priority Data
May 18, 2023 (KR) 10-2023-0064103

(51) Int. Cl. A63B 69/36; A63B 60/46; A63B 71/06

(52) U.S. Cl. CPC A63B 69/3632; A63B 60/46; A63B 71/0622; A63B 2071/0655; A63B 2214/00; A63B 2220/53

(57) ABSTRACT

The present invention relates to a golf lesson system and method using tactile stimulation, according to one embodiment, a golf lesson system comprise an impact sensor provided on a face of a clubhead and sensing a hitting point on the face where a golf ball has been hit, a stimulator divided into a plurality of stimulation sections and generating tactile stimulation and a processor controlling the stimulator to generate the tactile stimulation in the stimulation section corresponding to the hitting point.

The application claims the benefit under 35 USC § 119 (a) of Korean Patent Application No. 10-2023-0064103 filed on May 18, in the Korean Intellectual Property Office, the entire disclosure of which is incorporated herein by reference for all purpose.

### BACKGROUND

### 1. Field

The present invention relates to a golf lesson system using tactile stimulation and a golf lesson method using the same and, more specifically, to a golf lesson system and a golf lesson method capable of improving the efficiency of golf lesson by transmitting the information of position of a golf ball on the face of a clubhead using tactile stimulation such as electrical stimulation or vibration to a user who plays golf.

### 2. Description of the Related Art

In golf, since the carry distance and accuracy of the golf ball vary depending on which portion of the clubhead (head of a golf club) the golf ball is hit by, in order to play golf well, a technique of hitting the golf ball in a specific position of the clubhead is important depending on the situation.

Accordingly, users learn techniques to accurately hit golf ball to a desired position of the clubhead using golf academies or internet lectures. However, due to the nature of golf using a long golf club, it is quite difficult for a user to directly feel which part of the clubhead the golf ball is suitable for, and thus, various technologies and equipment capable of notifying the hitting point information have been developed.

One of the existing methods is to use a color-changing paper, which is a technology that attaches a color-changing paper when a strong impact is applied to a clubhead and checks a color change according to hitting a golf ball to check the hitting point.

In addition, there is also a visualization system in which a sensor or the like is attached to a clubhead to visually transmit information of the hitting point using a display screen or the like.

However, a method of using such color-changing paper or a method of visually transmitting the information on the display screen has no choice but to check the hitting point after the user plays golf. Therefore, a method of visually transmitting the hitting point information has disadvantages in that it is cumbersome to check the hitting point, and in that real-time feedback is impossible due to a time difference between a moment when a golf ball is hit and a moment checking the hitting point, thereby reducing learning efficiency.

Accordingly, there was still a need for a technology that could transmit the information of the hitting point in real time and enable immediate feedback.

### SUMMARY

A golf lesson system and a golf lesson method using tactile stimulation of the present invention are to enable a user to receive information on hitting point of a golf ball in real time by measuring the relative position of the clubhead and a golf ball when hitting the golf ball and transmitting the measured relative position to the user based on tactile stimulation such as electrical stimulation and vibration, and to enable immediate feedback, thereby leading to a significant increase in the effect of golf lesson.

According to one embodiment, a golf lesson system comprises an impact sensor provided on a face of a clubhead and configured to sense a hitting point at which a golf ball is hit on the face, a stimulator configured to divide into a plurality of stimulation sections and generate tactile stimulation and a processor configured to control the stimulator to generate the tactile stimulation in the stimulation section corresponding to the hitting point.

The tactile stimulation is an electrical stimulation.

The impact sensor further configured to measure the intensity of hitting and the stimulator is configured to generate electrical stimulation having different frequencies according to the intensity of hitting.

The impact sensor is further configured to measure a launch angle according to a hit of the golf ball and the stimulator is configured to generate electrical stimulation having different frequencies according to the launch angle.

The tactile stimulation is a vibration stimulation.

The impact sensor is further configured to measure the intensity of hitting and the stimulator is configured to generate vibration stimulation having different intensity of vibration according to the intensity of hitting.

The impact sensor is further configured to measure a launch angle according to a hit of the golf ball and the stimulator is configured to generate vibration stimulation having different intensities according to the launch angle.

The processor comprises a feedback device configured to set an arbitrary reference position on the face, and inform a user of an impact sensed at the reference position using another signal distinguished from the tactile stimulation when an impact is sensed at the reference position.

The impact sensor is configured to transmit a signal to the processor in the order of impact detection at the plurality of impact positions when impacts are detected at the plurality of impact sections during a process of hitting and the stimulator is configured to sequentially generate tactile stimulation in the stimulation section in order to correspond to the order of impact detection at the plurality of impact sections.

The golf lesson system further comprises a display configured to display, as visual information, whether an impact is applied to each of the impact sections.

According to another embodiment, a golf lesson method comprises sensing by an impact sensor provided on a face of a clubhead a hitting point at which a golf ball is hit on a face, generating by a stimulator a tactile stimulation by divided into a plurality of stimulation sections and controlling by a processor the stimulator to generate the tactile stimulation in the stimulation section corresponding to the hitting point.

The tactile stimulation is an electrical stimulation.

The sensing the hitting point further measures the intensity of hitting and the generating the tactile stimulation generates electrical stimulation having different frequencies according to the intensity of hitting.

The sensing the hitting point further measures a launch angle according to a hit of the golf ball and the generating the tactile stimulation generates electrical stimulation having different frequencies according to the launch angle.

The tactile stimulation is a vibration stimulation.

The sensing the hitting point further measures the intensity of hitting and the generating the tactile stimulation generates vibration stimulation having different intensity of vibration according to the intensity of hitting.

The sensing the hitting point further measures a launch angle according to a hit of the golf ball and the generating the tactile stimulation generates vibration stimulation having different intensities according to the launch angle.

The controlling the stimulator comprises setting an arbitrary reference position on the face and when an impact is sensed at the reference position on the face and informing a user of an impact sensed at the reference position using another signal distinguished from the tactile stimulation when an impact is sensed at the reference position.

The sensing the hitting point transmits a signal to the processor in the order of impact detection at the plurality of impact positions when impacts are detected at the plurality of impact sections during a process of hitting and the generating the tactile stimulation sequentially generates tactile stimulation in the stimulation section in order to correspond to the order of impact detection at the plurality of impact sections.

The golf lesson method further comprises displaying by a display, as visual information, whether an impact is applied to each of the impact sections.

According to one embodiment of the present invention that a golf lesson system and method using tactile stimulation, real-time feedback is enabled by transmitting information on the hitting point to a user using tactile stimulation, thereby providing a significantly elevated learning effect as compared with conventional methods.

### BRIEF DESCRIPTION OF THE DRAWINGS

FIGS. 1 and 2 are diagrams of a golf lesson system according to embodiments.

FIGS. 3 and 4 are examples of operation of a golf lesson system according to embodiments.

FIG. 5 is a view illustrating a stimulator 200 according to an embodiment.

FIGS. 6 to 10 are graphs of mapping functions according to embodiments.

FIG. 11 is a schematic diagram of a virtual table tennis experiment setup according to an embodiment.

FIG. 12 is a graph on a localization ratio of the hit point of the table tennis paddle according to an embodiment.

FIGS. 13 and 14 are views illustrating hitting point displayed on a table tennis paddle according to an embodiment.

FIG. 15 is a schematic diagram of a golf experiment setup according to an embodiment.

FIG. 16 to FIG. 21 are views showing the hitting points according to embodiments.

FIG. 22 is a graph showing a change in hitting accuracy for each group according to an embodiment.

FIG. 23 is a flowchart of a golf lesson method using tactile stimulation according to an embodiment.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences within and/or of operations described herein are merely examples, and are not limited to those set forth herein, but may be changed as will be apparent after an understanding of the disclosure of this application, except for sequences within and/or of operations necessarily occurring in a certain order. As another example, the sequences of and/or within operations may be performed in parallel, except for at least a portion of sequences of and/or within operations necessarily occurring in an order, e.g., a certain order. Also, descriptions of features that are known after an understanding of the disclosure of this application may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application. The use of the term “may” herein with respect to an example or embodiment, e.g., as to what an example or embodiment may include or implement, means that at least one example or embodiment exists where such a feature is included or implemented, while all examples are not limited thereto.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

The terminology used herein is for describing various examples only and is not to be used to limit the disclosure. The articles “a,” “an,” and “the” are intended to include the plural forms as well, unless the context clearly indicates otherwise. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof, or the alternate presence of an alternative stated features, numbers, operations, members, elements, and/or combinations thereof. Additionally, while one embodiment may set forth such terms “comprise” or “comprises,” “include” or “includes,” and “have” or “has” specify the presence of stated features, numbers, operations, members, elements, and/or combinations thereof, other embodiments may exist where one or more of the stated features, numbers, operations, members, elements, and/or combinations thereof are not present.

Due to manufacturing techniques and/or tolerances, variations of the shapes shown in the drawings may occur. Thus, the examples described herein are not limited to the specific shapes shown in the drawings, but include changes in shape that occur during manufacturing.

Unless otherwise defined, all terms, including technical and scientific terms, used herein have the same meaning as commonly understood by one of ordinary skill in the art to which this disclosure pertains specifically in the context on an understanding of the disclosure of the present application. Terms, such as those defined in commonly used dictionaries, are to be interpreted as having a meaning that is consistent with their meaning in the context of the relevant art and specifically in the context of the disclosure of the present application, and are not to be interpreted in an idealized or overly formal sense unless expressly so defined herein.

Hereinafter, a golf lesson system 1 using tactile stimulation and a golf lesson method using the same of the present invention will be described with reference to the drawings.

A golf lesson system 1 according to the present invention may sense a position where a golf ball is hit by a face 11 of a clubhead 10 when the golf ball is hit, using an impact sensor 100, and transmit tactile stimulation for each of a plurality of sections to a user using a stimulator 200 in response to the hitting point (impact position) where the golf ball is hit.

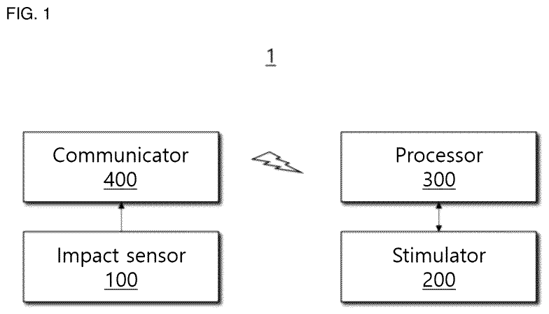

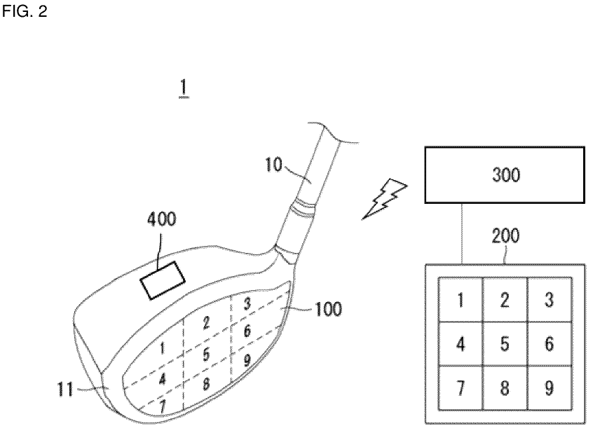

FIG. 1 is a block diagram of a golf lesson system 1 according to an embodiment of the present invention, and FIG. 2 is a view of a golf lesson system 1 according to an embodiment of the present invention.

Referring to FIGS. 1 and 2, a golf lesson system 1 of the present invention may include an impact sensor 100, a stimulator 200 and a processor 300.

The impact sensor 100 and the processor 300 of the golf lesson system 1 of the present invention may communicate with each other using the communicator 400. The impact sensor 100 and the processor 300 may communicate with each other in a wired system, but may preferably communicate with each other in a wireless system.

The impact sensor 100 will be described with reference to FIGS. 1 and 2.

The impact sensor 100 may be provided on the face 11 of the clubhead 10 to sense a hitting point on the face 11 where the golf ball is hit.

According to an embodiment, the impact sensor 100 may be provided on the face 11 of the clubhead 10 to divide the face 11 into a plurality of impact sections, and may sense the impact section in which an impact occurs due to the hitting a golf ball.

The impact sensor 100 may be attached to the face 11 of the clubhead 10, or may be integrally formed on the face 11. Here, the face 11 may mean a surface that the golf ball contacts when the golf ball is hit by clubhead 10, and the shape, angle, surface, and the like of the face 11 may vary according to the types of the clubhead 10. The impact sensor 100 according to the present invention may be formed in various shapes, angles, materials, and the like so as to be applied to the face 11 of the clubhead 10. The impact sensor 100 may be positioned to cover only a part of the face 11, and preferably, may be provided to cover the entire face 11.

The impact sensor 100 may sense an impact according to a hit of a golf ball. In this specification, sensing an impact by the impact sensor 100 may be understood as having the same meaning as detecting a contact. The impact sensor 100 may be implemented to include various types of contact detection sensors generally used to detect contact of an object. For example, the touch sensor may be a resistive type, a capacitive type, an electro-magnetic type (EM), or an optical type.

According to an embodiment, the impact sensor 100 may further measure the intensity of hitting according to the hitting of a golf ball. The impact sensor 100 may measure the intensity of hitting by calculating the impulse caused by hitting a golf ball. The impact sensor 100 may be implemented to include various types of impact measurement sensors generally used to measure an impulse when colliding with an object. For example, the impact sensor 100 may include an impact measurement sensor using an accelerometer measurement method, a piezoelectric element electrode measurement method, an inductive measurement method, a microphone and optical measurement method, and the like.

In addition, according to an embodiment, the impact sensor 100 may further measure an angle at which the golf ball flies (hereinafter, referred to as a launch angle) when the golf ball is hit. The launch angle at the time of hitting the golf ball is an important factor that affects the carry distance of the golf ball, and the launch angle of the golf ball may vary depending on the swing motion, the type of the clubhead 10, the hitting point on the face 11 of the golf ball, and the like.

The impact sensor 100 may measure the launch angle using a proximity object detection function to be described later.

The impact sensor 100 may be located on the face 11 to sense an object in a short distance the face surface using a proximity object sensing function. The impact sensor 100 may further include a proximity object detection function of a general proximity sensor to detect an object existing in a short distance from the face 11 on which the impact sensor 100 is located. Here, the short distance may mean a distance of about 1 cm from the face 11. The impact sensor 100 may measure a position on the face 11 closest to an object (e.g., a golf ball) and a distance from the object at each position on the face 11 using the proximity object detection function.

According to an embodiment, the proximity object detection function of the impact sensor 100 may be performed by emitting an electromagnetic field or electromagnetic waves and responding to a return signal. The proximity object detection function of the impact sensor 100 is not limited to the above-described method, and any technology used for a general proximity sensor may be used and implemented without limitation.

The impact sensor 100 may measure the launch angle using the position on the face 11 closest to a golf ball and a distance from the golf ball at each position on the face 11 measured by the proximity object detection function. Specifically, in the hitting process of the golf ball, the impact sensor 100 may acquire information on the position and distance of the golf ball approaching the face 11 before hitting, acquire information on the hitting point on the face 11 during hitting, and acquire information on the position and distance of the golf ball away from the face 11 after hitting. The impact sensor 100 may extract a trajectory when the golf ball is hit based on the above-described information (the approach/separation position and distance of the golf ball on the face), and may measure the launch angle of the golf ball.

According to another embodiment, the impact sensor 100 may be located on the face 11 to measure the launch angle of the golf ball with a method for calculating the launch angle by measuring an angle between a direction of a force where the golf ball hit and face 11 surface and hitting point of the golf ball on the face 11.

According to another embodiment, the golf lesson system 1 of the present invention may further include separate devices (e.g., an optical device for tracking a golf ball, etc.) capable of measuring the trajectory of a golf ball, thereby measuring the launch angle of the golf ball.

As a method of measuring the launch angle of the golf ball, a method generally used to measure the launch angle of the golf ball may be used, and is not limited to the above examples.

According to an embodiment, the impact sensor 100 may further measure the number of revolutions (or spin rate) of the ball. The impact sensor 100 may include a separate sensor capable of measuring the number of revolutions to measure the number of revolutions of the impacted golf ball, and may further include separate devices to measure spin, such as in the case of measuring a launch angle.

The impact measuring sensor and the contact detection sensor that may be used in the impact sensor 100 are not limited to the above example.

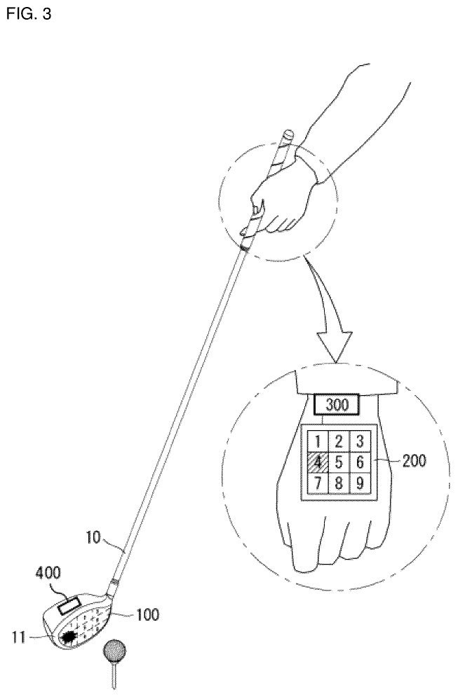

The impact sensor 100 may detect a hitting point on a face 11. According to an embodiment, the impact sensor 100 may divide the face 11 into a plurality of sections and sense an impact by dividing the sections. Specifically, the impact sensor 100 may be formed to divide the face 11 of the head of the golf club 10 into first to n-th impact sections to detect which impact section the impact is applied to. Referring to FIG. 3, as in the example of FIG. 3, the impact sensor 100 may divide the face 11 of the clubhead 10 into n impact sections and sense an impact applied to each impact section. For example, when the user hits the golf ball using the golf club, if the golf ball is hit in the fourth impact section of the face 11, the impact sensor 100 may sense that the golf ball has been hit in the fourth impact section. The impact sensor 100 is not limited to the example shown in the drawings, and may sense an impact by dividing the face into various shapes and numbers of impact sections.

The impact sensor 100 may sequentially sense a change in the impact occurrence position over time. When a golf ball is hit, the ball is not hit only on one point of the face 11, but may be hit through several positions. When an impact is generated while the golf ball moves along the face 11 while the hitting is in progress, the impact sensor 100 may sequentially sense a change in the hitting point.

According to an embodiment, when an impact is applied to the plurality of sections of the face 11, the impact sensor 100 may detect all impacts according to the plurality of sections. That is, when impacts are simultaneously sensed in several sections in a single hit, the impact sensor 100 may sense impacts on a plurality of sections rather than only an impact on one section.

According to an embodiment, the impact sensor 100 may sequentially sense a change in positions to which an impact is applied according to the movement of the golf ball. For example, when the user hits a golf ball using the golf club, the golf ball is first hit at a first position of the face 11, and then the golf ball impacts a second position, and then impacts a third position, the impact sensor 100 may sequentially sense impacts of the first, second, and third position.

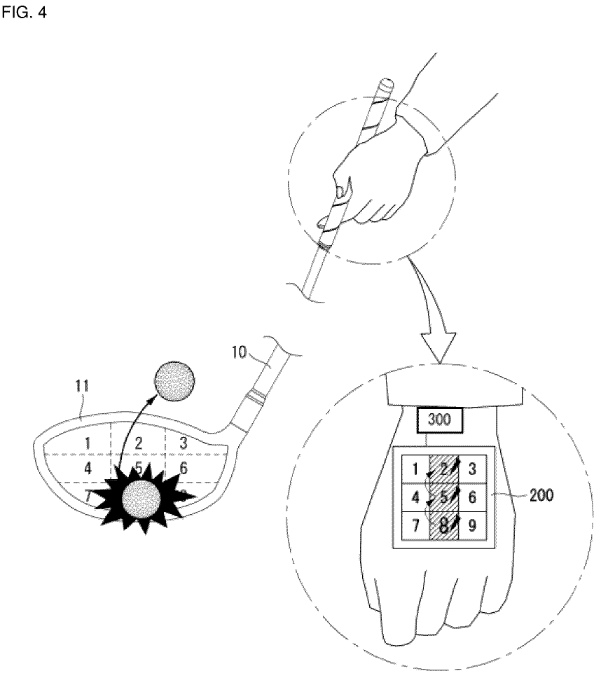

For example, referring to FIG. 4, when the golf ball is hit, the golf ball is initially hit by the impact section 8 of the face 11, the golf ball is moved up along the face 11 and contacts the impact section 5 of the face 11, and then moved up along the face 11 and contacts the impact section 2 of the face 11, the impact sensor 100 may sequentially sense the impacts of the impact sections 8, 5, and 2 in order.

The impact sensor 100 according to an embodiment of the present invention may be separated from the clubhead 10 and may be detachably attached to the face 11, or according to another embodiment, may be embedded in a clubhead 10.

According to an embodiment, when the impact sensor 100 is formed to be attached to or detached from the clubhead 10, the impact sensor 100 may be formed as a very thin sensor that may be attached to the face 11. Since the weight of the golf club is a factor that may affect golf ability, the impact sensor 100 attached to the clubhead 10 may be preferably formed in a very thin and light form.

In addition, the communicator 400 may be provided integrally with the impact sensor 100, but is not limited thereto, and may be separately provided at a position for minimizing a change in weight of the clubhead 10.

The stimulator 200 may receive various signals (e.g., impact sensing for each impact section, intensity of hitting, and launch angle) from the impact sensor 100 and/or the processor 300.

The stimulator 200 may provide tactile feedback on hitting a golf ball to the user by generating stimulation at a position corresponding to the impact position under the control of the processor 300.

The stimulator 200 may generate tactile stimulation by dividing into a plurality of stimulation sections.

According to an embodiment, the stimulator 200 may be divided into stimulation sections corresponding to the impact sections of the impact sensor 100 to generate tactile stimulation in the stimulation section corresponding to the impact section in which the impact occurs.

The stimulator 200 may be configured to be attached to a part of a human body. For example, the stimulator 200 may be formed to be attached to the back of a human hand, fingers, a palm, a wrist, an arm, and the like. The body part to which the stimulator 200 is attached is preferably the above-described hand or arm, but is not limited thereto.

The stimulator 200 may generate tactile stimulation. Here, the tactile stimulation may mean various stimulation that may be felt with tactile sensations of humans and animals. For example, the tactile stimulation may be largely classified into a mechanical stimulation and an electrical stimulation. The mechanical stimulation may include pressure stimulation, contact stimulation, vibration stimulation, and the like, and the electrical stimulation may include alternating-current electrical stimulation. In addition, the tactile stimulation may include a temperature stimulation such as a thermal stimulation. The tactile stimulation that may be generated by the stimulator 200 of the present invention is not limited to the above examples and may include various stimulation that may be felt with tactile sensation.

The stimulator 200 may include a stimulation generating device 250 required for generating a tactile stimulation. The stimulator 200 may adjust a stimulation section in which a stimulation is generated by adjusting the stimulation generating device 250 that generates the stimulation.

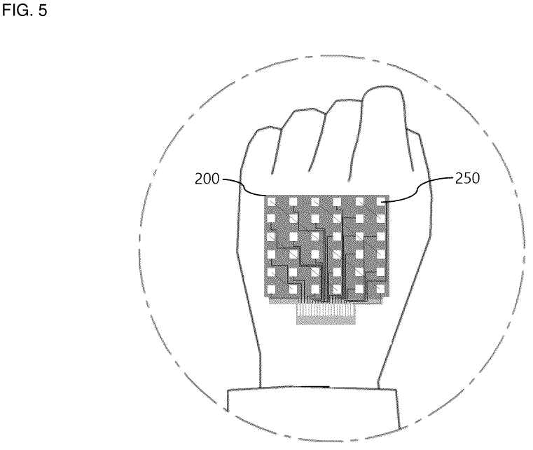

FIG. 5 is a view illustrating a stimulator 200 according to an embodiment. As shown in the example of FIG. 5, the stimulator 200 may include a plurality of stimulation generating devices 250. For example, when the tactile stimulation is a pressure stimulation, the stimulator 200 may include a pressure generating device for generating the pressure stimulation. In addition, when the tactile stimulation is a vibration stimulation, the stimulator 200 may include a vibration generating device for generating a vibration stimulation. In addition, when the tactile stimulation is an electrical stimulation, the stimulator 200 may include an electric discharge device for generating an electrical stimulation. The devices for generating a tactile stimulation are not limited to the above examples, and devices that may be used to generate a corresponding tactile stimulation may be used without limitation.

The stimulation generating device 250 may be located on a stimulation surface of the stimulator 200 so as to apply tactile stimulation to a corresponding body part when the stimulator 200 is attached to the back of a hand, a palm, a finger, an arm, or the like body part. The stimulation surface may mean a surface where a tactile stimulation is generated in the stimulator 200. For example, when the stimulator 200 is formed to be attached to the back of a user's hand, the stimulation surface refers to a surface in contact with the back of the user's hand, and the stimulation generating device 250 may be located on the stimulation surface of the stimulator 200 to generate stimulation on the back of the user's hand. In addition, when the stimulator 200 is formed to be attached to the user's palm, the stimulation surface means a surface that touches the user's palm of the stimulator 200, and the stimulation generating device 250 may be positioned on the stimulation surface of the stimulator 200 so as to generate stimulation on the user's palm. However, this form is only an example of a preferred form of the position of the stimulation generating device 250, and the form of the stimulator 200 is not limited thereto.

According to an embodiment, the stimulator 200 may be formed in a form included in a glove. The stimulator 200 may be located on the back of the hand of the glove so that the stimulation surface of the stimulator 200 may be located on the back of the hand when the user wears the glove. However, as described above, the stimulation surface may be located at a body part such as the back of a hand, a palm, a hand part including a finger, and/or an arm. In the present embodiment, the case where the back of the hand is selected as the stimulation surface is described, but the body portion where the stimulation surface is located is not limited to the above example.

According to an embodiment, when generating electrical stimulation, the stimulator 200 may generate electrical stimulation while changing the frequency of the electrical stimulation. The electrical stimulation may have a frequency, and may be classified into low frequency, high frequency, ultra-high frequency, and medium-frequency electrical stimulation according to the magnitude of the frequency. The frequency of the electrical stimulation that may be used in the present invention may be generated in various ranges of frequencies that can be detected by the tactile sense of the human body, and preferably in the range of 10 to 80 Hz. This is because a human may generally sense a difference according to a change in frequency in real time at modulation speed of 1 to 10 bps in a range of 10 to 80 Hz. However, since a range in which a frequency difference may be sensed for each individual may vary, such a frequency range may vary depending on a body part to which the stimulator 200 of the present invention is attached and/or a user.

According to an embodiment, when generating electrical stimulation, the stimulator 200 may receive the intensity of hitting measured by the impact sensor 100 and generate electrical stimulation having different frequencies according to the intensity of hitting.

For example, when the intensity of hitting measured by the impact sensor 100 is relatively strong, the stimulator 200 may generate an electrical stimulation having a high frequency by increasing an electrical frequency in proportion thereto. On the other hand, when the intensity of hitting is relatively weak, the stimulator 200 may generate an electrical stimulation having a low frequency by reducing the electrical frequency in proportion thereto.

On the contrary, according to another embodiment, when the intensity of hitting measured by the impact sensor 100 is relatively strong, the stimulator 200 may generate an electrical stimulation having a low frequency by reducing the electrical frequency in inverse proportion thereto. On the other hand, when the intensity of hitting is relatively weak, the stimulator 200 may increase the electrical frequency in inverse proportion thereto to generate electrical stimulation having a high frequency.

According to an embodiment, the stimulator 200 may generate the tactile stimulation having stimulation intensity (frequency, intensity of vibration, etc.) corresponding to hitting information (intensity of hitting, launch angle, spin rate, etc.) of the golf ball based on the mapping function.

According to an embodiment, the stimulator 200 may customize a mapping function between the intensity of hitting and the frequency for each user. The stimulator 200 may generate an electrical stimulation having a different frequency according to the intensity of hitting based on a mapping function customized for each individual. In this way, the user can more effectively receive feedback on the intensity of hitting using an electrical stimulation in a frequency range that can be better detected by each individual.

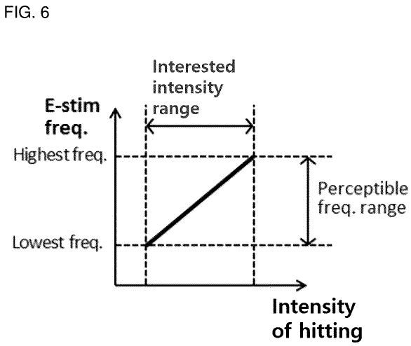

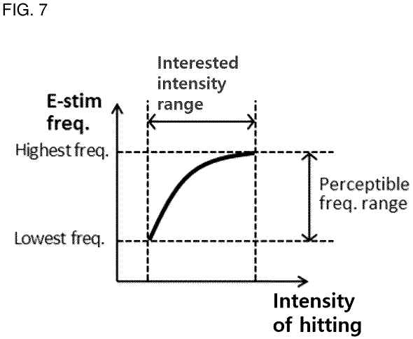

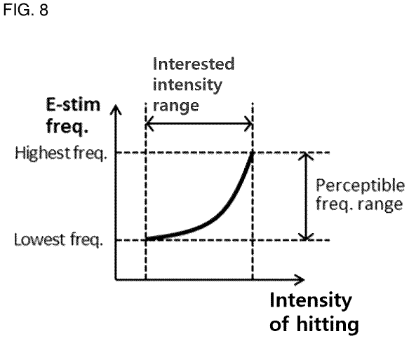

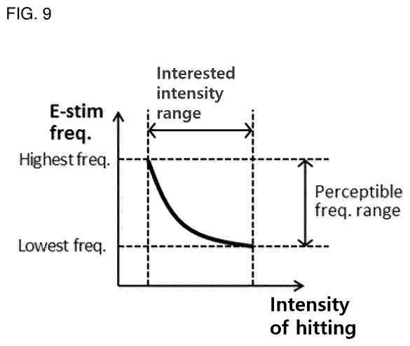

FIG. 6 to FIG. 10 are graphs of mapping functions according to the embodiments. The mapping functions of FIGS. 6 to 9 are a linear function, a logarithmic function, an exponential function, and an inverse proportional function, respectively. Referring to FIGS. 6 to 9, the stimulator 200 may calculate a frequency corresponding to the intensity of hitting measured using the mapping function, and may generate electrical stimulation based on the calculated frequency.

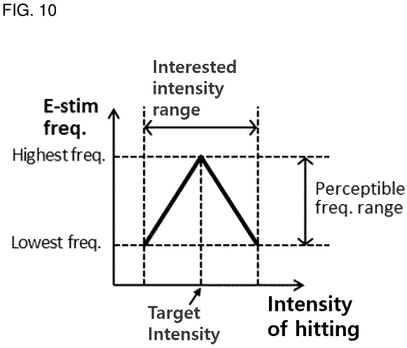

According to another embodiment, the stimulator 200 may use a mapping function that enables the intensity of hitting to correspond to a higher frequency as it approaches a preset target value. Referring to FIG. 10, the mapping function according to an embodiment may correspond to a higher frequency as the intensity of hitting is closer to a preset target value, and the stimulator 200 may generate an electrical stimulation having a higher frequency as the intensity of hitting is closer to the preset target value based on the mapping function.

According to an embodiment, when generating electrical stimulation, the stimulator 200 may generate electrical stimulation having a different frequency according to the launch angle of the golf ball. The stimulator 200 may receive information about the launch angle of the golf ball measured by the impact sensor 100 and generate electrical stimulation of a frequency proportional or inversely proportional to the launch angle. For example, when the shooting angle measured by the impact sensor 100 is relatively large, the stimulator 200 may generate electrical stimulation having a high frequency by increasing the electrical frequency in proportion thereto. On the other hand, when the launch angle is relatively small, the stimulator 200 may generate electrical stimulation having a low frequency by reducing the electrical frequency in proportion thereto.

According to an embodiment, the stimulator 200 may customize a mapping function between the launch angle and the frequency for each user. The stimulator 200 may generate electrical stimulation having a different frequency according to the launch angle based on a mapping function customized for each individual. In this way, the user can more effectively receive feedback on the launch angle using electrical stimulation in a frequency range that can be better detected by each individual. The stimulator 200 may utilize the above-described linear function, log function, exponential function, inverse proportional function, and the like as the mapping function, but is not limited thereto.

According to an embodiment, when generating vibration stimulation, the stimulator 200 may generate vibration stimulation while changing the intensity of the vibration. The vibration stimulation may have intensity, and the intensity of the vibration stimulation may be determined according to the amplitude and frequency of the vibration. The intensity of the vibration may have various ranges of amplitudes and frequencies, and may vary depending on the body part to which the stimulator 200 is attached.

According to an embodiment, when generating vibration stimulation, the stimulator 200 may receive the intensity of hitting measured by the impact sensor 100 and generate vibration stimulation having different intensities according to the intensity of hitting. For example, when the intensity of hitting measured by the impact sensor 100 is relatively strong, the stimulator 200 may generate vibration stimulation having a strong intensity in proportion thereto. On the other hand, if the intensity of hitting is relatively weak, the stimulator 200 may generate vibration stimulation of a weak strength in proportion thereto.

According to an embodiment, the stimulator 200 may customize a mapping function between the intensity of hitting and the intensity of vibration for each user. The stimulator 200 may generate vibration stimulation having a different intensity according to the intensity of hitting based on a mapping function customized for each individual. In this way, the user can more effectively receive feedback on the intensity of hitting using vibration stimulation in the intensity range that can be better detected by each individual. The stimulator 200 may utilize the above-described linear function, log function, exponential function, inverse proportional function, and the like as the mapping function, but is not limited thereto.

According to an embodiment, when generating vibration stimulation, the stimulator 200 may receive a launch angle measured by the impact sensor 100 and generate vibration stimulation having different intensities according to the launch angle. For example, when the shooting angle measured by the impact sensor 100 is relatively large, the stimulator 200 may generate vibration stimulation of a strong intensity in proportion thereto. On the other hand, if the launch angle is relatively small, the stimulator 200 may generate vibration stimulation of weak intensity in proportion thereto.

According to an embodiment, the stimulator 200 may customize a mapping function between the launch angle and the intensity of vibration for each user. The stimulator 200 may generate vibration stimulation having a different intensity according to the launch angle based on a mapping function customized for each individual. In this way, the user can more effectively receive feedback on the launch angle using vibration stimulation in the intensity range that can be better detected by each individual. The stimulator 200 may utilize the above-described linear function, log function, exponential function, inverse proportional function, and the like as the mapping function, but is not limited thereto.

According to an embodiment, the stimulator 200 may receive information on a distance to an object measured according to a proximity object detection function of the impact sensor 100, and generate tactile stimulation using a mapping function based on the received information. For example, the stimulator 200 may generate a tactile stimulation having a stimulation intensity inversely proportional to the distance from the object by using the inverse proportional function as a mapping function. Accordingly, the stimulator 200 may generate tactile stimulation having a higher intensity as the distance from the object decreases.

According to another embodiment, the stimulator 200 may receive the number of revolutions measured by the impact sensor 100 and generate tactile stimulation using a mapping function based on the received number of rotations. For example, the stimulator 200 may generate a tactile stimulation having a stimulation intensity logarithmically proportional to the number of revolutions of the golf ball using the logarithmic function as a mapping function.

According to an embodiment, each parameter (frequency, intensity of vibration, etc.) of the mapping function may be optimized according to individual characteristics. This may be performed according to the adjustment of the mapping function.

According to another embodiment, the generation of tactile stimulation having different stimulation intensities according to a mapping function by each stimulator 200 may be controlled by the processor 300.

The stimulator 200 may divide the sections and generate tactile stimulation for each section. Referring back to FIG. 5, the stimulator 200 may include a plurality of stimulation generating devices 250, and accordingly, may generate tactile stimulation to be divided into several stimulation sections and distinguished. The stimulator 200 may be attached to a part of the user's body, divide the stimulation surface to which the stimulator 200 is attached into a plurality of stimulation sections, and separately generate stimulation for each stimulation section.

According to an embodiment, the stimulator 200 may divide the stimulation surface into a plurality of stimulation sections to correspond to the division of the impact sensor 100 into a plurality of impact sections. Specifically, the stimulator 200 may divide the stimulation surface into first to n-th stimulation sections so that the impact sensor 100 corresponds to the division of the first to n-th impact sections. Referring back to FIG. 3, as the impact sensor 100 divides the impact position into sections 1 to 9, the stimulator 200 may also divide the stimulation surface into sections 1 to 9 corresponding to the impact sensor 100. Accordingly, the stimulator 200 may be formed by dividing the stimulation section so that the impact sensor 100 corresponds to the division of the impact section.

According to an embodiment, the impact section and the stimulation section may be divided such that relative positions thereof correspond to each other. That is, the relative position on the face 11 of the impact section and the relative position on the stimulation surface of the stimulation section may correspond. Accordingly, each stimulation section of the stimulator 200 may be divided to correspond to a relative position of each impact section of the impact sensor 100. For example, the first stimulation section of the stimulator 200 may correspond to the first impact section of the impact sensor 100, and the second stimulation section of the stimulator 200 may correspond to the second impact section of the impact sensor 100. In addition, the position of the stimulator 200 on the stimulation surface of the n-th impact section may be the same as the position of the impact sensor 100 on the face 11 of the n-th impact section.

According to an embodiment, the stimulator 200 may generate tactile stimulation in the stimulation section in response to detecting an impact in each impact section of the impact sensor 100. For example, when an impact is sensed in the first impact section of the impact sensor 100, the stimulator 200 may generate tactile stimulation in the first stimulation section corresponding thereto. In this way, the user hitting the ball may know at which position of the face 11 the ball is hit with the position of the stimulation generated from the stimulator 200.

According to an embodiment, when the impact sensor 100 senses impact for a plurality of impact sections during the process of the hitting, the stimulator 200 may receive a signal in an impact sensing order of the plurality of impact sections and generate tactile stimulation on the stimulation sections to correspond to the impact sensing order of the plurality of impact sections. Referring back to the example of FIG. 4, when the impact is detected in the order of the eighth impact section, the fifth impact section, and the second impact section of the impact sensor 100 during the process of the hitting, the stimulator 200 may generate tactile stimulation in the order of the eighth stimulation section, the fifth stimulation section, and the second stimulation section.

The processor 300 may receive a signal from the impact sensor 100 according to impact sensing, and may be connected to the stimulator 200 in a wired or wireless system to control the stimulator 200 to generate tactile stimulation in a plurality of stimulation sections corresponding to a hitting point.

As described above, the processor 300 may control the stimulator 200 so that a tactile stimulation is generated in a stimulation section corresponding to the hitting point when the golf ball is hit.

According to an embodiment, the processor 300 may receive the hitting point sensed by the impact sensor 100 as a signal, allocate it to a plurality of pre-divided impact sections, and then control the stimulator 200 to generate a tactile stimulation in a stimulation section corresponding thereto.

According to an embodiment, the processor 300 may control the stimulator 200 to generate a tactile stimulation having a stimulation intensity corresponding to the mapping function based on the above-described mapping function according to the intensity of hitting or the launch angle.

The processor 300 may further include a feedback device (not shown). The feedback device may set an arbitrary reference position on the face, and when the impact sensor 100 senses an impact at the reference position, may inform the user of this using another signal distinguished from the tactile stimulation. Here, the arbitrary reference position may be a position arbitrarily set by the user among the plurality of impact sections, or may be a position set by the device itself. The reference position may be at least one of a plurality of impact sections, and a plurality of reference positions may be set.

The feedback device may inform the user that the impact is detected at the set reference position of the impact sensor 100 using another signal distinguished from the tactile stimulation. Here, the other signal may be a voice signal, a visual signal, or another tactile signal distinguished from the tactile signal generated by the stimulator 200 of the present invention. Accordingly, the golf lesson system 1 may include a device (e.g., a speaker, a display, etc.) for generating each signal.

According to an embodiment, the other signals may be implemented by making the number, intensity, and the like of tactile stimulation generated by the stimulator 200 different. For example, when a hitting point is sensed at the reference position, the stimulator 200 may generate tactile stimulation several times in the stimulation section corresponding to the reference position to inform the user that the impact is applied to the reference position.

Another signal generated by the feedback device is to be distinguished from the tactile signal to be notified to the user, and various signals may be used without being limited to the above-described examples.

The golf lesson system 1 of the present invention may further include a display (not shown). The display may receive a signal from the impact sensor 100 and notify the user of whether a signal is generated in each impact section. In addition, when an impact is detected in the reference section, it may be notified to the user using the display. According to an embodiment, the display may be implemented in the form of a mobile device, and the mobile device may be implemented in a form that can be worn on the user's body. For example, the user may check the hitting information in real time by wearing a mobile device including a display on the wrist.

The golf lesson system 1 of the present invention may further include a wireless transceiver/receiver (not shown). The wireless transceiver may be electrically connected to devices (impact sensors, stimulators, etc.) included in the golf lesson system 1 to enable signal transmission of each device, or may be included in each device to enable signal transmission of each device.

The golf lesson system 1 of the present invention may receive feedback with tactile stimulation on which position of the face 11 of the clubhead 10 the ball is hit when the golf ball is hit using the impact sensor 100 and the stimulator 200.

Hereinafter, the learning effect of the golf lesson system 1 by tactile stimulation according to the present invention will be described with reference to Experimental Examples 1 and 2.

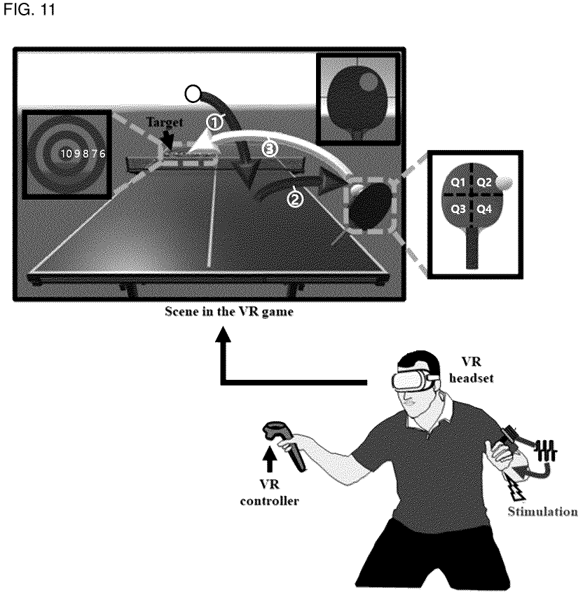

FIG. 11 is a schematic diagram of a virtual table tennis experiment setup for Experimental Example 1.

In Experimental Example 1, in table tennis similar to golf, experiments based on a learning method using visual stimulation and tactile stimulation of the present invention were performed, with the aim of fitting a table tennis ball to a specific position of a table tennis paddle for healthy people.

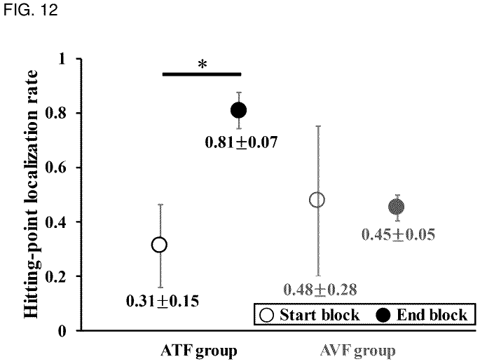

The experimental result of Experimental Example 1 will be described with reference to FIGS. 12 to 14.

Augmented Tactile Feedback (ATF) group refers to a group learned using tactile stimulation, and Augmented Visual Feedback (AVF) group refers to a group learned using visual stimulation. Start block and End block refer to the measurement results before and after the experiment of each group.

FIG. 12 is a graph on a localization ratio of the hit point of the table tennis paddle.

According to FIG. 12, in the case of the AVF group learned using visual stimulation, the localization ratio of the hitting point before and after learning decreased from 0.48 to 0.45. On the other hand, in the case of the ATF group learned using tactile stimulation, the result was that the localization ratio of the hitting point before and after learning increased significantly from 0.31 to 0.81. Therefore, it can be seen that when learning using tactile stimulation, there is a remarkable effect in improving the learning efficiency of localization of hitting point.

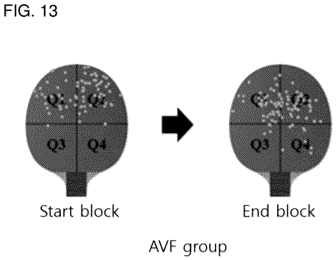

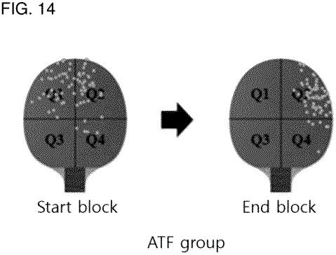

FIGS. 13 and 14 are views illustrating hitting point displayed on a table tennis paddle.

According to FIGS. 13 and 14, in the case of the AVF group, the hitting point before and after learning became rather unclear, and in the case of the ATF group, it was confirmed that the hitting point before and after learning was formed at a predetermined position, and thus the hitting point was confirmed to be accurate. As a result, it can be seen that there is a remarkable effect on improving the accuracy of hitting point when learning using tactile stimulation.

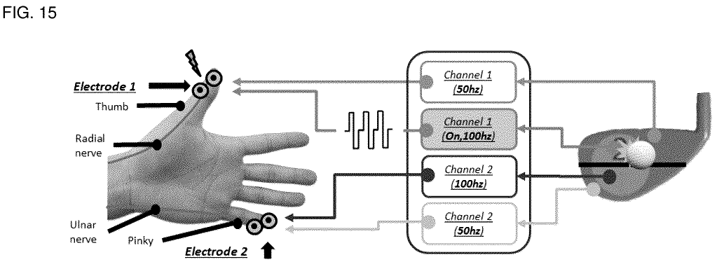

FIG. 15 is a schematic diagram of a golf experiment setup for Experimental Example 2.

Experimental Example 2 aims to fit a golf ball to a specific position on the face 11 of the head of the clubhead 10 for healthy people in golf, and experiments based on a learning method using visual stimulation and the tactile stimulation of the present invention were performed.

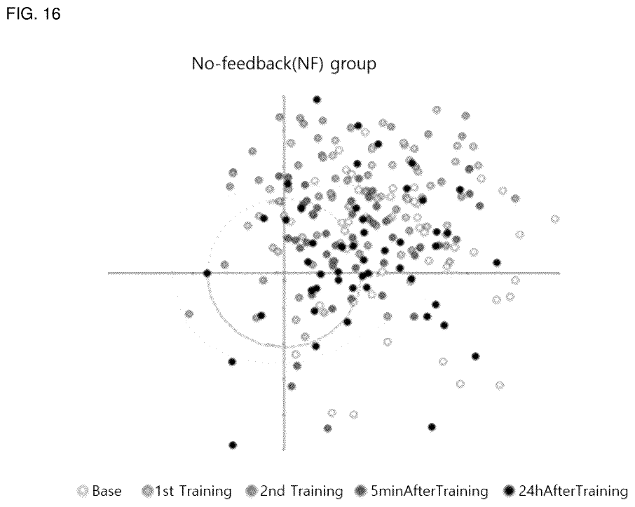

Experimental results of Experimental Example 2 will be described with reference to FIGS. 16 to 22.

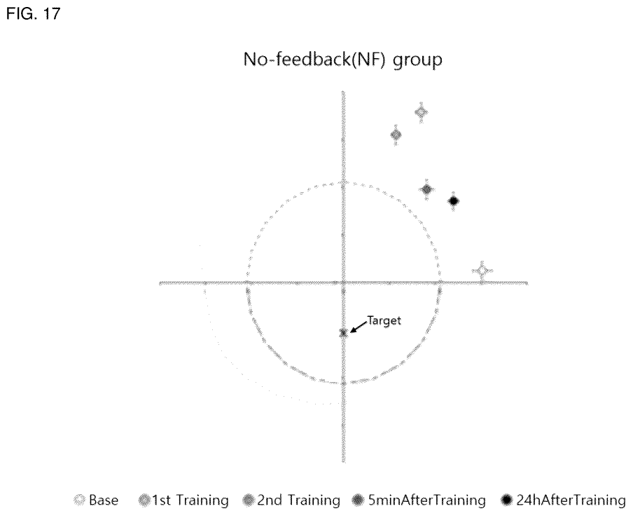

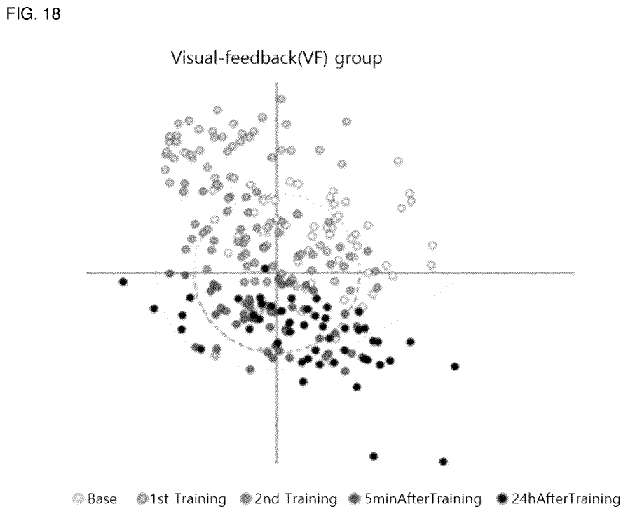

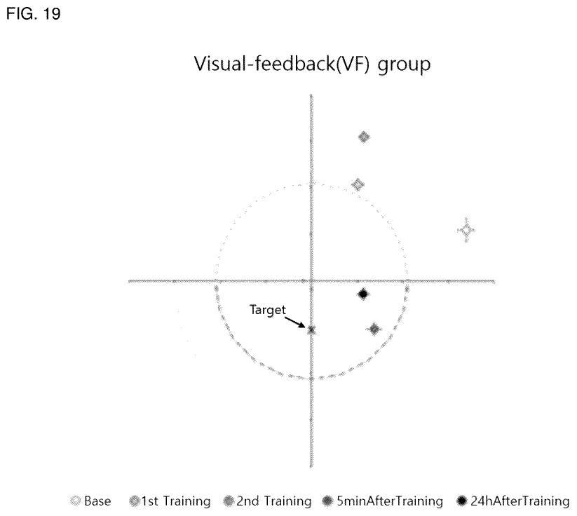

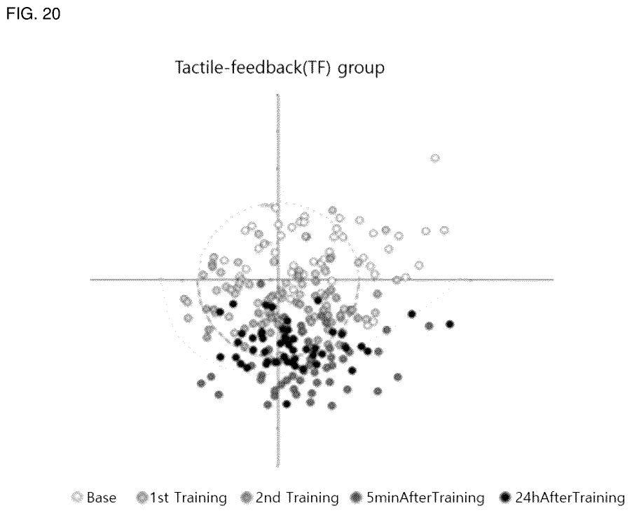

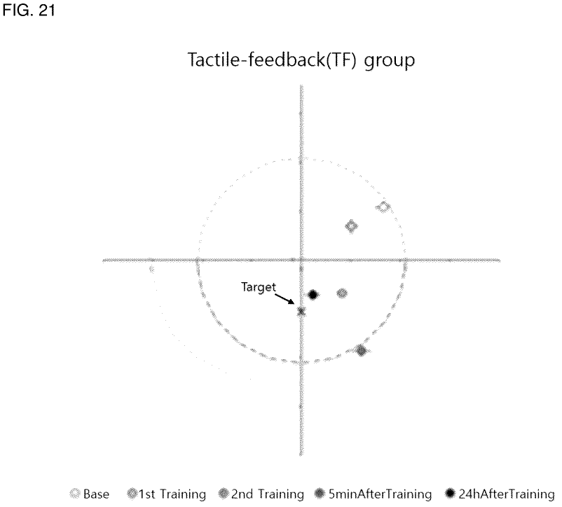

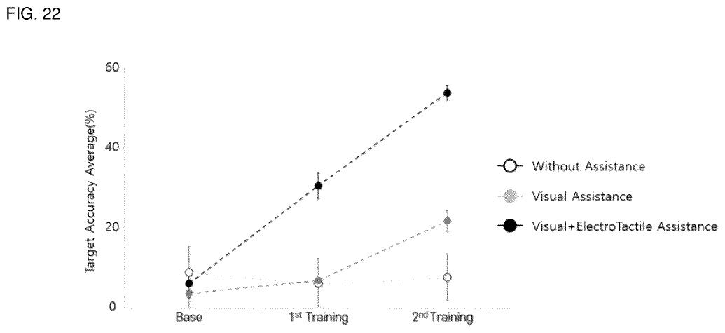

FIG. 16 to FIG. 21 are views showing the hitting points according to an experiment. Specifically, FIGS. 16 and 17 show experimental results of a group that does not perform feedback, FIGS. 18 and 19 show experimental results of a group that performs feedback using visual stimulation, and FIGS. 20 and 21 show experimental results of a group that performs feedback using visual stimulation and tactile stimulation of the present invention. In addition, FIG. 22 is a graph showing a change in hitting accuracy for each group.

Referring to FIGS. 16 to 21, in the case of the group that did not provide feedback, it can be seen that the training was not effective, as the hitting point where the golf ball was hit was not close to the target position even in the late training (black dot) compared to the initial training (white dot). On the other hand, in the case of the group that performs feedback using visual stimulation and tactile stimulation of the present disclosure, it can be confirmed that the hitting point is fitted in the late training (black dot) is significantly closer to the target compared to the initial training (white dot).

Referring to FIG. 22, it can be confirmed that the target accuracy average is most significantly increased as the training progresses in the case of the group that has performed feedback using visual stimulation and tactile stimulation of the present invention.

Accordingly, as a result of Experimental Example 2, it may be seen that there is a remarkable effect on improving the accuracy of hitting points when learning using tactile stimulation.

In conclusion, referring to Experimental Examples 1 and 2, it can be expected that the golf lesson system using tactile stimulation of the present invention can have a remarkable effect on improving the accuracy of hitting a golf ball of a user.

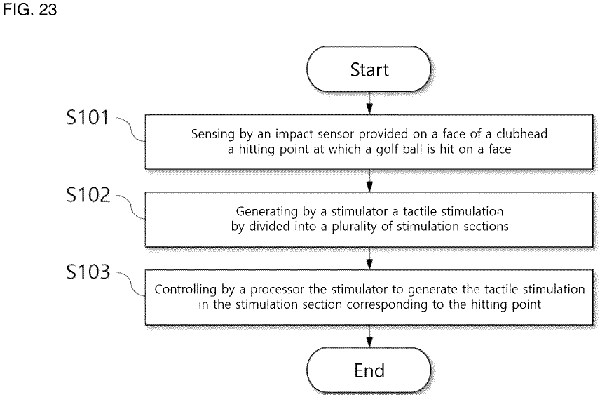

The golf lesson method using tactile stimulation according to the present invention will be described with reference to FIG. 23.

FIG. 23 is a flowchart of a golf lesson method using tactile stimulation according to an embodiment.

According to an embodiment, the golf lesson method using tactile stimulation may include an impact sensing step S101 of sensing a hitting point on the face 11 of the clubhead 10 using the impact sensor 100.

In addition, the golf lesson method using tactile stimulation may include a stimulation generating step S102 of generating tactile stimulation by dividing into a plurality of stimulation sections using the stimulator 200.

In addition, the golf lesson method using tactile stimulation may include a control step S103 of controlling the stimulator 200 to generate tactile stimulation in the stimulation section corresponding to the impact section using the processor.

According to one embodiment, the tactile stimulation is an electrical stimulation.

According to one embodiment, the sensing the hitting point further measures the intensity of hitting and the generating the tactile stimulation generates electrical stimulation having different frequencies according to the intensity of hitting.

According to one embodiment, the tactile stimulation is a vibration stimulation.

According to one embodiment, the sensing the hitting point further measures the intensity of hitting and the generating the tactile stimulation generates vibration stimulation having different intensity of vibration according to the intensity of hitting.

According to one embodiment, the controlling the stimulator comprises setting an arbitrary reference position on the face and when an impact is sensed at the reference position on the face and informing a user of an impact sensed at the reference position using another signal distinguished from the tactile stimulation when an impact is sensed at the reference position.

According to one embodiment, the sensing the hitting point transmits a signal to the processor in the order of impact detection at the plurality of impact positions when impacts are detected at the plurality of impact sections during a process of hitting and the generating the tactile stimulation sequentially generates tactile stimulation in the stimulation section in order to correspond to the order of impact detection at the plurality of impact sections.

According to one embodiment, the golf lesson method further comprises displaying by a display, as visual information, whether an impact is applied to each of the impact sections.

Although the processor 300 and the stimulator 200 are distinguished from each other for convenience in the present specification, the processor 300 and the stimulator 200 may be configured as the same device to perform the above operations.

Various embodiments of the golf lesson system 1 and method using tactile stimulation have been described, but the system or method is not limited to the above-described embodiments. Various other devices or methods that a person having ordinary knowledge in the art may modify and modify based on the above-described embodiments may also be one embodiment of the above-described golf lesson system 1 or golf lesson method. For example, if the described method(s) are performed in a different order than described, and/or the described component(s) of the system, structure, device, circuit, etc. are combined, connected or combined or combined in a different form than described, or replaced or replaced by other components or equivalents, etc., it may be one embodiment of the above-described golf lesson system 1 and/or golf lesson method.

In addition to the above and all drawing disclosures, the scope of the disclosure is also inclusive of the claims and their equivalents, i.e., all variations within the scope of the claims and their equivalents are to be construed as being included in the disclosure.

What is claimed is:

1. A golf lesson system comprising:
an impact sensor provided on a face of a clubhead and configured to sense a hitting point at which a golf ball is hit on the face;
a stimulator configured to divide into a plurality of stimulation sections and generate tactile stimulation; and
a processor configured to control the stimulator to generate the tactile stimulation in the stimulation section corresponding to the hitting point.

2. The golf lesson system of claim 1,
wherein the tactile stimulation is an electrical stimulation.

3. The golf lesson system of claim 2,
wherein the impact sensor is further configured to measure the intensity of hitting, and
the stimulator is configured to generate electrical stimulation having different frequencies according to the intensity of hitting.

4. The golf lesson system of claim 2,
wherein the impact sensor is further configured to measure a launch angle according to a hit of the golf ball, and
the stimulator is configured to generate electrical stimulation having different frequencies according to the launch angle.

5. The golf lesson system of claim 1,
wherein the tactile stimulation is a vibration stimulation.

6. The golf lesson system of claim 5,
wherein the impact sensor is further configured to measure the intensity of hitting, and
the stimulator is configured to generate vibration stimulation having different intensity of vibration according to the intensity of hitting.

7. The golf lesson system of claim 5,
wherein the impact sensor is further configured to measure a launch angle according to a hit of the golf ball, and
the stimulator is configured to generate vibration stimulation having different intensities according to the launch angle.

8. The golf lesson system of claim 1,
wherein the processor comprises a feedback device configured to set an arbitrary reference position on the face, and inform a user of an impact sensed at the reference position using another signal distinguished from the tactile stimulation when an impact is sensed at the reference position.

9. The golf lesson system of claim 1,
wherein the impact sensor is further configured to transmit a signal to the processor in the order of impact detection at the plurality of impact positions when impacts are detected at the plurality of impact sections during a process of hitting, and
the stimulator is configured to sequentially generate tactile stimulation in the stimulation section in order to correspond to the order of impact detection at the plurality of impact sections.

10. The golf lesson system of claim 1,
the golf lesson system further comprising:
a display configured to display, as visual information, whether an impact is applied to each of the impact sections.

11. A golf lesson method comprising:
sensing by an impact sensor provided on a face of a clubhead a hitting point at which a golf ball is hit on a face;
generating by a stimulator a tactile stimulation by divided into a plurality of stimulation sections; and
controlling by a processor the stimulator to generate the tactile stimulation in the stimulation section corresponding to the hitting point.

12. The golf lesson method of claim 11,
wherein the tactile stimulation is an electrical stimulation.

13. The golf lesson method of claim 12,
wherein the sensing the hitting point further measures the intensity of hitting, and
the generating the tactile stimulation generates electrical stimulation having different frequencies according to the intensity of hitting.

14. The golf lesson method of claim 12,
wherein the sensing the hitting point further measures a launch angle according to a hit of the golf ball, and
the generating the tactile stimulation generates electrical stimulation having different frequencies according to the launch angle.

15. The golf lesson method of claim 11,
wherein the tactile stimulation is a vibration stimulation.

16. The golf lesson method of claim 15,
wherein the sensing the hitting point further measures the intensity of hitting, and
the generating the tactile stimulation generates vibration stimulation having different intensity of vibration according to the intensity of hitting.

17. The golf lesson method of claim 15,
wherein the sensing the hitting point further measures a launch angle according to a hit of the golf ball, and
the generating a tactile stimulation generates vibration stimulation having different intensities according to the launch angle.

18. The golf lesson method of claim 11,
wherein the controlling the stimulator comprising:
setting an arbitrary reference position on the face and,
informing a user of an impact sensed at the reference position using another signal distinguished from the tactile stimulation when an impact is sensed at the reference position.

19. The golf lesson method of claim 11,
wherein the sensing the hitting point transmits a signal to the processor in the order of impact detection at the plurality of impact positions when impacts are detected at the plurality of impact sections during a process of hitting, and
the generating the tactile stimulation sequentially generates tactile stimulation in the stimulation section in order to correspond to the order of impact detection at the plurality of impact sections.

20. The golf lesson method of claim 11,
the golf lesson method further comprising:
displaying by a display, as visual information, whether an impact is applied to each of the impact sections.
